(19) United States
(12) Patent Application Publication
(10) Pub. No.: US 2017/0000414 A1
(43) Pub. Date: Jan. 5, 2017

(54) Use of Blood Flow Parameters to Monitor or Control the Dosing of Anti-Platelet Agents

(71) Applicant: RHEOVECTOR, LLC, Camden, NJ (US)

(72) Inventor: Daniel J. Cho, Wayne, PA (US)

(21) Appl. No.: 15/114,279

(22) PCT Filed: Feb. 4, 2015
(86) PCT No.: PCT/US15/14420

§ 371 Date: Jul. 26, 2016

Related U.S. Application Data
(60) Provisional application No. 61/935,495, filed on Feb. 4, 2014.

(51) Int. Cl. A61B 5/00; A61B 5/026; A61B 5/02

(52) U.S. Cl. CPC A61B 5/4848; A61B 5/02035; A61B 5/026; A61B 5/4839

(57) ABSTRACT

The invention comprises a method of using blood flow parameters to optimize the therapeutic efficacy of an antiplatelet agent in the treatment of a disease or disorder associated with abnormal hemodynamic thrombogenicity in a subject.

### CROSS-REFERENCE TO RELATED APPLICATIONS

This application claims priority to U.S. Provisional Application Ser. No. 61/935,495, filed Feb. 4, 2014, the content of which is incorporated by reference herein in its entirety.

### BACKGROUND OF THE INVENTION

Platelets are discoid cells found in large numbers in blood, which are important for blood coagulation and hemostasis. Upon activation by various stimuli like thrombin, thromboxane and ADP, platelets change into a spheroid shape with filopodia, degranulate and aggregate. Platelet activation is important for hemostasis and underlies various pathological conditions such as unstable angina pectoris, myocardial infarction, stroke, and coagulopathies. One of the physiological agents that activate platelets is thrombin, a serine protease. Thrombin mediates its action through the activation of protease activated receptors (PARs), including PAR-1 and PAR-4. See, e.g., U.S. Pat. No. 6,444,695.

While platelets are essential for normal blood clotting, overactive platelets can contribute to pathology. Platelet activation is the cause or a significant contributor to several vascular and non-vascular diseases. Platelet-dependent arterial thrombosis is known to trigger most heart attacks and strokes (Khan M L et al. Nature. Aug. 13, 1998; 394(6694):690-4).

A variety of anti-clotting agents have been developed in the art, including antiplatelet agents and anticoagulants. Antiplatelet drugs developed in the art include aspirin, which is the most common anti-clotting drug; glycoprotein IIb/IIIa inhibitors, such as abciximab (ReoPro™, Eli Lilly & Co.), eptifibatide (Integrilin™, Schering Plough Corp., Millenium Pharmaceuticals, and Glaxo Smith Kline), tirofoban (Aggrastat™, Merck & Co., Inc.) and lamifiban; and inhibitors of ADP-induced platelet activation, including thienopyridines, such as clopidogrel (Plavix™, Sanofi-Bristol Myers Squibb) and ticlopidine (Ticlid™, Roche Laboratories). Anticoagulants developed in the art include heparin, such as standard unfractionated heparin, and low molecular weight heparins (LMWHs), such as ardeparin, dalteparin, enoxaparin and tinzaparin.

A series of antiplatelet agents have been developed over the past several years based on different mechanisms of action. The most widely used agent in antiplatelet therapy is aspirin, which irreversibly inhibits cyclooxygenase-1 and thereby affects the thromboxane pathway. Although not optimally efficacious, treatment with aspirin remains the standard therapy against which new therapeutics are compared and judged.

Other drugs like the phosphodiesterase inhibitors dipyridamole and cilostazol, as well as the vitamin K antagonists (warfarin), are marketed but do not show all desirable features for such drugs. Three intravenously applicable, potent GPIIb/IIIa receptor antagonists (abciximab, eptifibatide, and tirofiban) blocking platelet aggregation are available on the market. Various orally active GPIIb/IIIa antagonists (e.g. sibrafiban, xemilofiban or orbofiban) have not been successful in clinical development so far.

Adenosine 5′-diphosphate (ADP) is a key mediator in platelet activation and aggregation interfering with two platelet ADP receptors P2Y₁ and P2Y₁₂. Antagonists of the platelet ADP receptor have been identified and display inhibition of platelet aggregation and antithrombotic activity. The most effective antagonists known so far are the thienopyridines ticlopidine, clopidogrel and CS-747 (prasugrel), which have been used clinically as antithrombotic agents.

As a high level of platelet function inhibition and platelet aggregation inhibition has been reported to be associated with a decrease in the incidence of major adverse cardiac events, the question becomes whether clinicians should uniformly use aggressive and costly antiplatelet strategies without direct individual assessment of hemodynamic status in patients with thrombotic or ischemic disorders. A simple and rapid method of identifying patients who would benefit from platelet aggregation inhibition or platelet antagonist therapy is desirable. Further, a simple, reliable and rapid method of monitoring treatment would be useful in optimizing safety and efficacy of antiplatelet therapy.

Thus, there is a need in the art for a rapid, simple test for predicting thrombotic vascular problems as well as to monitor safety and efficacy of antiplatelet therapy. The present invention satisfies this need in the art.

### SUMMARY OF THE INVENTION

The invention provides a method for determining the efficacy of an antiplatelet agent in a subject. In one embodiment, the method comprises monitoring at least one blood flow parameter in a subject; administering an antiplatelet agent at a first dose to a subject; and targeting a new dose of the agent if the value of the at least one blood flow parameter crosses a predetermined threshold value.

In one embodiment, the at least one blood flow parameter is selected from the group consisting of whole blood viscosity, low shear viscosity, and yield stress of blood.

In one embodiment, the subject is suffering from a vascular disease or disorder.

In one embodiment, the vascular disease or disorder is selected from the group consisting of a disorder of hemodynamic thrombogenicity, a thrombotic disorder, ischemia, acute coronary syndrome, stroke, ischemic complications of peripheral vascular disease, deep vein thrombosis, myocardial infarction, coronary artery disease, cerebrovascular disease, peripheral arterial disease, diabetes mellitus, diabetic retinopathy, atrial fibrillation, congestive heart failure, pulmonary embolism, and any combination thereof.

In one embodiment, the antiplatelet agent is ticagrelor.

In one embodiment, the at least one blood flow parameter is whole blood viscosity further wherein the whole blood viscosity value of less than about 13 cP when measured at a low shear rate of 5 sec⁻¹ indicates the efficacy of the antiplatelet agent for the subject.

The invention also provides a method for improving the safety and efficacy of an antiplatelet agent in a subject. In one embodiment, the method comprises measuring a whole blood viscosity of the subject to obtain a first value; administering an antiplatelet agent to the subject; measuring a whole blood viscosity of the subject to obtain a second value wherein a value of less than about 13 cP but greater than about 6 cP when measured at a low shear rate of 5 sec⁻¹, indicates the hemorrhagic safety and thrombogenic efficacy of the antiplatelet agent for the subject; and administering a subsequent dose of the antiplatelet agent to the subject to attain the value of less than about 12 cP when measured at a low shear rate of 5 sec⁻¹.

The invention also provides a method for improving the safety and efficacy of an antiplatelet agent in a subject. In one embodiment, the method comprises measuring a whole blood viscosity of the subject to obtain a first viscosity value; administering an antiplatelet agent to the subject; measuring a whole blood viscosity of the subject to obtain a second viscosity value; determining a value for a difference between the first viscosity value and the second viscosity value; comparing the value for the difference to a threshold value; targeting a whole blood viscosity value for the subject if the value of the difference has crossed the threshold value; and administering a subsequent dose of the antiplatelet agent to attain the targeted whole blood viscosity.

### DETAILED DESCRIPTION

Systems and methods are described herein to evaluate a candidate medication as it relates to a subject's cardiovascular health. A processing component is employed to measure a first value of one or more markers, which are associated with a circulatory system of each subject that is to receive the candidate medication. The candidate medication is administered to each subject and a second value of one or more markers are measured subsequent to the administration as of the candidate medication. Continued testing of the candidate medication can be continued dependent upon the change in the one or more markers.

The present invention relates to the discovery that blood flow parameters (e.g., blood viscosity) can be used to monitor and/or control the dosing of an antiplatelet agent. In one embodiment, the invention comprises a method of using blood flow parameters to optimize the therapeutic efficacy of an antiplatelet agent in the treatment of a disease or disorder associated with thrombotic vascular dysfunction in a subject. Blood flow parameters include but are not limited to circulating blood viscosity, absolute viscosity, effective viscosity, low shear viscosity, high shear viscosity, shear rate of circulating blood, work of heart, contractility of heart, thrombogenicity, platelet aggregation, lubricity, red blood cell deformability, thixotropy, yield stress, coagulability, coagulation time, agglutination, clot retraction, clot lysis time, sedimentation rate and prothrombin rate.

In one embodiment, the present invention uses blood viscosity as a marker to assess one or more of early apprehension of susceptibility to vascular pathological events, response to platelet antagonist therapies, generate individualized antiplatelet regimens, monitor subjects once therapy has been initiated, control dosing of an antiplatelet agent, and the like.

Included in the invention are methods for assessing blood viscosity in a subject, including the methods of assessing blood flow parameters, specifically those incorporating whole blood viscosity measurements, as set forth in U.S. Pat. Nos. 6,152,888, 6,193,667, 6,200,277, 6,261,244, 6,322,524, 6,659,965, 6,796,168, 6,805,674, and 6,907,772, all of which are incorporated herein by reference. Accordingly, in one embodiment, the invention provides the use of blood flow parameters as a tool for monitoring or controlling dosing of an antiplatelet agent, whereby efficacy of the antiplatelet agent may be improved.

In one embodiment, the invention provides methods of using blood viscosity values to determine a baseline status, and correlating the change in the blood viscosity status following receipt of an antiplatelet therapy to the presence or absence of a platelet-affected disease state. The inventive methods are useful for predicting susceptibility to a broad range of vascular pathologies, including, acute coronary syndrome, stroke, ischemic complications of peripheral vascular disease, deep vein thrombosis, myocardial infarction, coronary artery disease, cerebrovascular disease, peripheral arterial disease, diabetes mellitus, diabetic retinopathy, atrial fibrillation, congestive heart failure, pulmonary embolism, a disorder of hemodynamic thrombogenicity, a thrombotic disorder, ischemia or other related disease states.

In one embodiment, the method of optimizing dose of an antiplatelet agent comprises the steps of: (a) determining the viscosity of the blood of the subject; (b) reducing the viscosity of the blood by administering an antiplatelet agent to the subject; and (c) re-determining the viscosity of the blood of the subject to verify the reduction in the viscosity.

Another aspect of the present invention is directed to methods for predicting response to platelet aggregation inhibition or antiplatelet therapy in subjects, including the steps of determining the blood viscosity of a subject's blood, using the blood viscosity values to determine baseline hemodynamic thrombogenicity status, and correlating high baseline hemodynamic thrombogenicity status with appropriateness of antiplatelet therapy. Such methods are useful for, identifying subjects who would be most likely to benefit from more aggressive antiplatelet regimens, predicting the efficacy of such treatments in individual subjects, controlling dosing of an antiplatelet agent, whereby efficacy of the antiplatelet agent may be improved.

Also provided in the invention is a method for monitoring a subject during an antiplatelet therapy regimen. In one embodiment, the method comprises determining the blood viscosity value in the subject's blood, and using the blood viscosity value to determine whether the levels of hemodynamic thrombogenicity are reduced by the platelet antagonist therapy, thereby indicating the production of a therapeutic effect.

Thus, according to one embodiment of the invention, whole blood viscosity can be used to monitor and assess the efficacy and safety of an antiplatelet therapy regimen in a subject under treatment for the correction of one or more of the abnormal hemodynamic thrombogenicity, platelet function and platelet aggregation. In one embodiment, whole blood viscosity can be used to target the dose of the antiplatelet therapy regimen used to treat the disease or disorder associated with elevated thrombogenicity as well as abnormal platelet function and aggregation in the subject.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About,” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

By “ameliorate,” “modulate,” or “decrease” is meant a lessening or lowering or prophylactic prevention of the detrimental effect of the disorder in the subject receiving the therapy, thereby resulting in “protecting” the subject.

“Antiplatelet” and “platelet antagonist” therapies are those that inhibit platelet activity, including but are not limited to aggregation, accumulation, adhesion, and/or cohesion.

“Antiplatelet agents” or “platelet inhibitors” or “platelet aggregation inhibitors” are agents that block the formation of blood clots by preventing the aggregation of platelets. There are several classes of antiplatelet agents based on their activities, including, GP IIb/IIIa antagonists, such as abciximab (ReoPro™) eptifibatide (Integrilin™), and tirofiban (Aggrastat™); P2Y.sub.12 receptor antagonists, such as clopidogrel (Plavix™), ticlopidine (Ticlid™), cangrelor, ticagrelor, and prasugrel; phosphodiesterase III (PDE III) inhibitors, such as cilostazol (Pletal™), dipyridamole (Persantine™) and Aggrenox™ (aspirin/extended-release dipyridamole); thromboxane synthase inhibitors, such as furegrelate, ozagrel, ridogrel and isbogrel; thromboxane A2 receptor antagonists (TP antagonist), such as ifetroban, ramatroban, terbogrel, (3-{6-[(4-chlorophenylsulfonyl)amino]-2-methyl-5,6,7,8-tetrahydronaphth-1-yl}propionic acid (also known as Servier S18886, by de Recherches Internationales Servier, Courbevoie, France); thrombin receptor antagonists, such as SCH530348 (having the chemical name of ethyl (1R,3aR,4aR,6R,8aR,9S,9aS)-9-((E)-2-(5-(3-fluorophenyl)pyridin-2-yl)vinyl)-1-methyl-3-oxodod-ecahydronaphtho[2,3-C]furan-6-ylcarbamate, by Schering Plough Corp., New Jersey, USA, described in US20040192753A1 and US2004/0176418A1 and studied in clinical trials, such as A Multicenter, Randomized, Double-Blind, Placebo-Controlled Study to Evaluate the Safety of SCH 530348 in Subjects Undergoing Non-Emergent Percutaneous Coronary Intervention with ClinicalTrials.gov Identifier: NCT00132912); P-selectin inhibitors, such as 2-(4-chlorobenzyl)-3-hydroxy-7,8,9,10-tetrahydrobenzo[H]quinoline-4-carbo-xylic acid (also known as PSI-697, by Wyeth, N.J., USA); and non-steroidal anti-inflammatory drugs (NTHES), such as acetylsalicylic acid (Aspirin), resveratrol, ibuprofen (Advil™, Motrin™), naproxen (Aleve™, Naprosyn™), sulindac (Clinoril™), indomethacin (Indocin™), mefenamate, droxicam, diclofenac (Cataflam™, Voltaren™), sulfinpyrazone (Anturane™), and piroxicam (Feldene™) Among the NTHES, acetylsalicyclic acid (ASA), resveratrol and piroxicam are preferred. Some NTHES inhibit both cyclooxygenase-1 (cox-1) and cyclooxygenase-2 (cox-2), such as aspirin and ibuprofen. Some selectively inhibit cox-1, such as resveratrol, which is a reversible cox-1 inhibitor that only weakly inhibits cox-2. Beta blockers and calcium channel blockers, which are described below, also have a platelet-inhibiting effect.

The term “thrombogenicity” is the tendency of blood to coagulate, clot, or result in the formation of a thrombus. The term “thrombogenic” refers to a factor that causes or encourages the development of a thrombus and the coagulation of blood.

The term “assessing” includes any form of measurement, and includes determining if an element is present or not. The terms “determining,” “measuring,” “evaluating,” “assessing,” and “assaying” are used interchangeably and include quantitative and qualitative determinations. Assessing may be relative or absolute. “Assessing the presence of” includes determining the amount of something present, and/or determining whether it is present or absent.

The term “hemodynamic” refers to the mechanical or physical forces involved in the flow of blood. These forces include the viscous forces and inertial forces of circulating blood. Insofar as blood flows within a lumen or elastic tubular segment having a specific diameter, as is the case in the cardiovascular system, hemodynamic forces also include shear and strain, reflecting the interaction between circulating blood and the vessel lumen. The former relates to the tangential frictional forces of blood flow along the lumen, while the latter relates to the circumferential tension applied by blood flow against the lumen.

As used herein, the term “cardiovascular disease” or “CVD,” generally refers to heart and blood vessel diseases, including atherosclerosis, coronary heart disease, cerebrovascular disease, and peripheral vascular disease. Cardiovascular disorders are acute manifestations of CVD and include myocardial infarction, stroke, angina pectoris, transient ischemic attacks, and congestive heart failure. Cardiovascular disease, including atherosclerosis, usually results from the build-up of cholesterol, inflammatory cells, extracellular matrix and plaque.

As used herein, the term “coronary heart disease” or “CHD” refers to atherosclerosis in the arteries of the heart causing a heart attack or other clinical manifestation such as unstable angina.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “dose”, “dosage”, “unit dose”, “unit dosage”, “effective dose”, “effective amount” and related terms refer to physically discrete units that contain a predetermined quantity of the active agent, calculated to produce a desired therapeutic effect.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, “platelet-affected disease” refers to a disorder characterized by abnormal levels of platelet activation.

“Sample” or “biological sample” as used herein means a biological material isolated from a subject. The biological sample may contain any biological material suitable for detecting the desired analytes, and may comprise cellular and/or non-cellular material obtained from the subject.

A “therapeutic” treatment is a treatment administered to a subject who exhibits signs or symptoms of pathology, for the purpose of diminishing or eliminating those signs or symptoms.

As used herein, “treating a disease or disorder” means reducing the frequency or severity with which a sign or symptom of the disease or disorder is experienced by a patient.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

An “effective amount” of a delivery vehicle is that amount sufficient to effectively bind or deliver a compound.

As used herein, the term “efficacy” refers to the maximal effect (Eₘₐₓ) achieved within an assay.

The phrases “vascular disease,” “vascular disorder,” “vascular condition,” “vascular pathology,” and the like, refer to bodily states affecting the channels and tissue that carry body fluids, such as, but not limited to stroke, deep vein thrombosis (DVT), myocardial infarction, coronary artery disease, cerebrovascular disease, peripheral arterial disease, diabetes mellitus, diabetic retinopathy, atrial fibrillation, congestive heart failure, acute coronary syndrome, stroke, pulmonary embolism, and ischemic complications of peripheral vascular disease.

As used herein, “yield stress” equals the applied shear stress (tangential frictional force) that must be exceeded in order to make a structured fluid flow. Yield stress also refers to the minimum force required for blood to flow.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention includes compositions and methods useful in subjects at risk of or suffering from a disease or condition associated with abnormal hemodynamic thrombogenicity (e.g., the tendency for blood to clot or coagulate based primarily on the physical rather than chemical properties influenced by blood flow). In one embodiment, the invention includes compositions and methods useful to predict or determine a subject's response to one or more antiplatelet agents. In certain embodiments, the antiplatelet agents include, but are not limited to, glycoprotein IIb/IIIa inhibitor such as abciximab, eptifibatide, and tirofiban; ADP receptor/P2Y12 inhibitors such as thienopyridines (clopidogrel, prasugrel, ticlopidine) and ticagrelor; prostaglandin analogues (PGI2) such as beraprost, prostacyclin, iloprost, and treprostinil; COX inhibitors such as acetylsalicylic acid/aspirin, aloxiprin, carbasalate calcium, indobufen, and triflusal; thromboxane synthase inhibitors such as dipyridamole, picotamide; receptor antagonist such as terutroban; phosphodiesterase inhibitors such as cilostazol, dipyridamole, triflusal or others such as cloricromen and ditazole.

It will be appreciated that aspects of the invention are useful in determining a subject's suitability for a treatment regime, preferably in determining a subject's suitability to prophylactic or therapeutic treatment with an antiplatelet agent, including but is not limited to a platelet aggregation inhibitor such as an antagonist of the adenosine diphosphate receptor P2Y₁₂ (e.g., Ticagrelor). However, the invention should not be limited to only Ticagrelor. Rather, the invention is applicable to other antiplatelet therapy including for example, clopidogrel, cangrelor, prasugrel, abciximab, elinogrel, eptifibatide, tirofiban; heparin and derivatives thereof.

P2Y₁₂ receptors play an active role in platelet activation. In the normal state, when blood vessels are damaged, platelet activation mediated by P2Y₁₂ receptors play an important role to arrest bleeding at the site of injury. In a diseased state, platelet activation leads to vascular occlusion and ischemic damage. Thus, P2Y₁₂ receptors antagonists play a key role in antiplatelet therapy in assisting to prevent among other things coronary artery disease.

In addition to coronary artery disease, the invention is applicable to other diseases such as peripheral artery disease (PAD). PAD is a condition in which there is an obstruction of an artery in the arms or legs. In some instances, PAD diagnosis begins with a physical examination. A physician or qualified person assesses for weak pulses in the extremity. The ankle-brachial index (ABI) test is also performed. The ABI test compares the blood pressure in an individual's foot to the blood pressure in your arms to determine how well blood is flowing. This inexpensive test takes only a few minutes and can be performed as part of a routine exam. Normally, the ankle pressure is at least about 90 percent of the arm pressure, but with severe narrowing it may be less than about 50 percent.

In one embodiment, whole blood viscosity value in a subject is used as a marker to determine a subject's response to one or more antiplatelet agents. In another embodiment, blood viscosity value in a subject is used as a marker to optimize the dosing of the one or more antiplatelet agents, whereby efficacy of the antiplatelet agent may be improved.

In one embodiment, the invention comprises a method for optimizing therapeutic efficacy of an antiplatelet agent in treating a disease or disorder associated with abnormal hemodynamic thrombogenicity, comprising administering one or more doses of an antiplatelet agent in the subject in which blood viscosity after administering an initial dose or doses of the antiplatelet agent significantly decreases to a value that corresponds to a therapeutic outcome. In one embodiment, the low shear blood viscosity value measured at shear rate of 5 sec⁻¹ corresponding to a therapeutic outcome is around 6-14 centipoises (cP). In another embodiment, the low shear blood viscosity value measured at shear rate of 5 sec⁻¹ corresponding to a therapeutic outcome is around 7-12 cP. In another embodiment, the low shear blood viscosity value measured at shear rate of 5 sec⁻¹ corresponding to a therapeutic outcome is around 8-11 cP. In one embodiment, the low shear blood viscosity value measured at shear rate of 5 sec⁻¹ corresponding to a therapeutic outcome is around 12 cP.

In one embodiment, the blood viscosity value after administering an initial dose or doses of the antiplatelet agent are those at any timing after administering the initial dose.

In one embodiment, the timing prior to the administration of the antiplatelet agent can be any timing prior to administering the initial dose, but the timing immediately prior to administering the initial dose is preferable. Therefore, a baseline blood viscosity value can be calculated at any time prior to the administration of the antiplatelet agent. On the other hand, the timing after administering the initial dose or doses of the antiplatelet agent can be any timing after administration of the initial dose or doses of the antiplatelet agent. Therefore, a test blood viscosity value can be calculated at any time after to the administration of the antiplatelet agent. The test blood viscosity value can be used in combination with any other criteria to confirm the antiplatelet effects of the antiplatelet agent by conventional measurement or a conventional evaluation method. Exemplary criteria for assessing antiplatelet effects include but are not limited to complete blood count (CBC), fibrinogen, globulins, lipid profile, ankle/toe brachial index, pulse volume recording, and the like. The measurement of blood viscosity and other assessment criteria is not limited to one time, but may be performed at a plurality of times after the initial dose of the antiplatelet agent.

In one embodiment, the method of optimizing therapeutic efficacy of an antiplatelet agent comprises the steps of administering an antiplatelet agent to a subject having a disease or disorder associated with abnormal hemodynamic thrombogenicity; and determining a blood viscosity value in the subject, where the blood viscosity value is not significantly lowered compared to the blood viscosity prior to receiving the antiplatelet agent indicates a need to increase the amount of antiplatelet agent subsequently administered to the subject and where a blood viscosity value that is significantly lowered indicates a potential to decrease the amount of the antiplatelet agent subsequently administered to the subject. In one embodiment, the amount of the antiplatelet agent can be adjusted to an amount that generates a blood viscosity value of about 6-14 cP when measured at a low shear rate of 5 sec⁻¹. In another embodiment, the amount of the antiplatelet agent can be adjusted to an amount that generates a blood viscosity value of about 7-12 cP when measured at a low shear rate of 5 sec⁻¹. In another embodiment, the amount of the antiplatelet agent can be adjusted to an amount that generates a blood viscosity value of about 8-11 cP when measured at a low shear rate of 5 sec⁻¹. In another embodiment, the amount of the antiplatelet agent can be adjusted to an amount that generates a blood viscosity value of about 12 cP when measured at a low shear rate of 5 sec⁻¹.

The present invention also provides a method of reducing hemorrhagic toxicity associated with antiplatelet agent treatment of a disease or disorder associated with abnormal hemodynamic thrombogenicity comprising the steps of administering an antiplatelet agent to a subject; and determining a blood viscosity value in the subject, where a blood viscosity value less than a predetermined blood viscosity value associated with toxic level indicates a need to decrease the amount of the antiplatelet agent subsequently administered to the subject, thereby reducing toxicity associated with antiplatelet agent treatment of the disease or disorder. In one embodiment, the blood viscosity value associated with a predetermined toxic level of antiplatelet agent can correspond, for example, to a level of about 7 cP when measured at a low shear rate of 5 sec⁻¹, 6 cP when measured at a low shear rate of 5 sec⁻¹, or 5 cP when measured at a low shear rate of 5 sec⁻¹.

In one embodiment, the invention provides a method of optimizing therapeutic efficacy and reducing toxicity associated with an antiplatelet agent treatment of a disease or disorder associated with abnormal hemodynamic thrombogenicity. The method includes the steps of administering an antiplatelet agent to a subject; determining a blood viscosity value in the subject, where a blood viscosity value that is not significantly lowered compared to a predetermined blood viscosity value associated with a minimal therapeutic level indicates a need to increase the amount of the antiplatelet agent subsequently administered to the subject, thereby increasing therapeutic efficacy; where a blood viscosity value less than a predetermined blood viscosity value associated with a toxic level of the antiplatelet agent indicates a need to decrease the amount of the antiplatelet drug subsequently administered to the subject, thereby reducing toxicity associated with the antiplatelet agent treatment of the disease or disorder.

In one embodiment, the invention includes a method of determining whether a subject is given a minimal therapeutic dose of an antiplatelet agent by assessing the blood viscosity value of the subject following receipt of the antiplatelet agent. In one embodiment, the method of determining whether a subject is given a minimal therapeutic dose of an antiplatelet agent is useful for indicating to the clinician a need to monitor a subject for therapeutic efficacy and to adjust the dose of the antiplatelet agent, as desired. For example, in a subject having less than a minimal therapeutic level of an antiplatelet agent based on the blood viscosity value and who also presents as unresponsive to an antiplatelet therapy or having poor responsiveness to antiplatelet therapy as measured by minimal or no effect on a sign or symptom of the disease being treated, one skilled in the art can determine that the dosage of an antiplatelet should be increased. However, if it is determined that a subject has less than a predetermined minimal therapeutic level of an antiplatelet agent but is responsive to antiplatelet therapy based on blood viscosity value, the current dose of the antiplatelet agent can be maintained. Based on measuring blood viscosity value following administration of the antiplatelet agent and assessing the responsiveness of the subject to the antiplatelet therapy, one skilled in the art can determine whether the antiplatelet dose should be maintained, increased, or decreased.

In one embodiment, an optimal dosage of the antiplatelet agent can be determined based on the correlation of the blood viscosity value and therapeutic outcome of the subject receiving the antiplatelet agent. In accordance with other embodiments, as a result of the method, the subject's whole blood viscosity is reduced an average of at least about 10% when measured at a low shear rate of 5 sec⁻¹.

It will be understood by those skilled in the art that the dosage regimen includes the quantity of drug administered (dose) and the frequency of administration (dosing interval). It will also be appreciated that various methods of drug delivery are available such as intravenous or intraperitoneal injection or the like and that often drugs will be introduced by infusion. In one embodiment, the dosing profile of an antiplatelet drug is determined by its effect on whole blood viscosity in the recipient subject.

Another aspect of the present invention is directed to methods for predicting response to antiplatelet therapy in subjects with a hemodynamically thrombogenic disease state, including the steps of determining the blood viscosity value in subject, using the blood viscosity value to determine the appropriateness of the antiplatelet therapy. In one embodiment, blood viscosity value is used to determine the appropriateness of the dosing of the antiplatelet agent. In one embodiment, the dosing of the antiplatelet agent can be optimized based on the blood viscosity value. For example, a preferred range of blood viscosity can be determined by correlating the blood viscosity value with other criteria used to assess efficacy of the antiplatelet agent including but not limited to complete blood count (CBC), fibrinogen, globulins, lipid profile, ankle/toe brachial index, pulse volume recording, and the like.

In one embodiment, blood viscosity value can be used to identify subjects who would be most likely to benefit from antiplatelet regimens and predict the efficacy of such treatments in individual subjects.

The invention also provides methods for monitoring subjects during an antiplatelet therapy regimen comprising determining the blood viscosity values in subject blood samples over time, and determining whether these blood viscosity values decrease over time, indicating that the levels of hemodynamic thrombogenicity in these subjects are reduced by the platelet antagonist therapy (i.e., indicating a therapeutic effect).

### Measurement of Blood Viscosity

Blood is a heterogeneous fluid consisting mainly of plasma and a suspension of red blood cells. Red cells tend to aggregate when the flow shear rates are low, while increasing shear rates break these formations apart, thus reducing blood viscosity. This results in two blood properties, shear thinning and yield stress. In healthy large arteries, blood can be successfully approximated as a homogeneous fluid since the vessel size is much greater than the size of particles and shear rates are sufficiently high that particle interactions may have a negligible effect on the flow.

In a normal, healthy blood vessel, a physiologic range of wall shear stresses are maintained by mechanical forces produced by blood flow. Wall shear stress in a blood vessel, i.e., the frictional force per unit area acting tangentially to the arterial wall, is determined by the product of shear rate and blood viscosity. The shear rate is defined as the velocity gradient within the lumen and is determined by the first derivative of flow velocity with respect to the distance from the vessel wall. Viscosity is a fluid's resistance to flow.

The viscosity of plasma, a Newtonian fluid, does not depend on characteristics of its flow. Whole blood, on the other hand, behaves as a non-Newtonian fluid, and its viscosity depends on its shear rate. Specifically, whole blood is more viscous at low shear rates and becomes relatively less viscous at higher shear rates. The shear rate dependent aspect of blood viscosity poses a challenge to accurately determining the wall shear stress in a specific blood vessel.

Blood viscosity has been shown to be independently associated with the major risk factors for cardiovascular disease including: hypertension (Devereux et al, 2000. Am J Cardiol 85:1265-8; Fowkes et al., 1993 Eur Heart 14:597-601; Letcher et al., 1981 Am J Med 1981 70:1195-202); hyperlipidema (positive correlation with total cholesterol, LDL-cholesterol and triglyceride; negative correlation with HDL-cholesterol) (Stamos et al., 1999 Atherosclerosis 146:161-5; Sloop et al., 1997 Clin Sci 92:473-9; Rosenson et al., 1996 Clin Chem 42:1189-95; Rosenson et al., 2002 Atherosclerosis 2002; 161:433-9; Lowe et al., 1992 Circulation 85:2329-31; de Simone et al., 1990 Circulation 81:107-17); diabetes, insulin resistance syndrome and obesity (de Simone et al., 1990 Circulation 81:107-17; Jax et al., 2009 Cardiovasc Diabetol 8:48; Tamariz et al., 2008 Am J Epidemiol 168:1153-60; Hoieggen et al., 1998 J Hypertens 16:203-10; Ernst et al., 1986 Atherosclerosis 59:263-9); tobacco smoking (Ernst et al., 1995 J Cardiovasc Risk 2:435-9; Levenson et al., 1987 Arteriosclerosis 7:572-7; Ernst et al., 1988 Arteriosclerosis 8:385-8; Lowe et al., 1980 Scott Med J 25:13-7); male gender (de Simone et al., 1990 Circulation 81:107-17; Kameneva et al., 1999 Clin Hemorheol Microcirc 21:357-63; Fowkes et al., 1994 Arterioscler Thromb 14:862-8); and aging (de Simone et al., 1990 Circulation 81:107-17; Lowe et al., 1980 Scott Med J 25:13-7; Coppola et al., 2000 Arch Gerontol Geriatr 31:35-42). Atherosclerosis, which is the underlying cause of most cardiovascular disease events, develops at specific arterial sites, such as the outer walls of bifurcation sites. Site-specific development of atherosclerotic lesions has long provided pathophysiological support for the critical role of hemodynamic forces in the progression of atherosclerosis (Zarins et al., 1983 Circ Res 53:502-14). The fact that atherosclerotic plaques do not develop uniformly throughout the vasculature but at specific arterial sites has underscored the importance of studying mechanical forces of blood flow and their interactions with the endothelial wall. A key hemodynamic force is wall shear stress, defined as the tangential friction applied by blood flow against the endothelial wall, and is determined by blood viscosity (Malek et al., 1999 JAMA 282:2035-42; Frangos et al., 1999 Arch Surg 134:1142-9; Davies et al., 2009 Nat Clin Pract Cardiovasc Med 6:16-26; Kensey et al., 2003 Curr Med Res Opin. 19:587-96; Baskurt et al., 2003 Semin Thromb Hemost 29:435-50).

The invention relates to systems and methods to utilize at least viscosity of blood to influence the use of medication administered to a subject to treat blood related diseases and disorders. This viscosity of blood and be combined with other cardiovascular data to gauge the risk for potential adverse cardiovascular risk for an individual subject using a particular medication, within a clinical practice setting, and with other systems and methods to evaluate blood viscosity as a marker to alter treatment of a subject. In one example, the embodiments herein can be based upon an analysis of an antiplatelet agent (e.g., ticagrelor). However, any candidate medications are within the scope of the subject invention.

Some medical conditions and medications cause fluid retention. Fluid retention in turn can increase intravascular volume and pressure. When fluid retention occurs, some of the fluid is retained in the intravascular compartment. With normal vascular tone, and within limits, the cardiovascular system can vasodilate in order to accommodate the extra volume, and often there is no change in blood pressure (BP). BP can increase, however, if the cardiovascular system does not vasodilate enough to accommodate the extra volume and pressure, as occurs when vascular tone is compromised secondary to atherosclerotic disease as an example. As a result, cardiac output (CO) can increase following an increase in intravascular volume. When CO increases, SV, heart rate, or both, will increase. Change in either BP or SV can influence the velocity of blood flow. The subject embodiments can be employed to calculate the effect of fluid retention upon velocity of blood flow.

Accordingly, the invention includes methods for assessing blood viscosity in a subject, including the methods of using blood flow parameters as set forth in U.S. Pat. Nos. 6,152,888, 6,193,667, 6,200,277, 6,261,244, 6,322,524, 6,659,965, 6,796,168, 6,805,674, and 6,907,772, all of which are incorporated herein by reference. In one embodiment, the invention provides the use of blood flow paramaters as a tool for monitoring or controlling dosing of an antiplatelet agent, whereby efficacy of the antiplatelet agent may be improved.

For example, blood viscosity can be measured by tube-type viscometers, rotational viscometers, microfluidic channel-type viscometers, porous bed viscometers, ultrasonographic viscometers, catheter-type viscometers, cantilever-type viscometers, microelectronic viscometers, and other functionally similar instruments. In one embodiment, an automated scanning capillary (tube-type) viscometer is used, consisting of two main components: a height detection system to measure height variations in the two riser columns in a U-shaped disposable tube, connected by a horizontal capillary tube. Blood is first introduced into the first riser column through a stopcock valve. Once the first column is filled with blood, the blood then is permitted to travel through the horizontal capillary tube and is introduced into the second column using the computer-controlled three-way stopcock, allowing the blood in the first column to fall and the blood in the second column to rise. The pressure drop is determined from the height difference measurement (i.e., (ρg[h₁−h₂]) in the two riser columns, while the volume flow rate Q(t) is mathematically determined using the first derivative of the height with respect to time, dh/dt. Since the diameter and length of the capillary tube are known values, this scanning capillary viscometer is able to determine the blood viscosity from the pressure drop and flow rate data. Note that the geometry of the U-tube controls the flow rate, and thus, the shear rate, at the capillary tube from an initial maximum value to almost zero as the two fluid levels in the riser columns approach each other. In this way, the blood viscosity values measured can be obtained over a wide range of shear rates, i.e., from 1000 to 1 s⁻¹.

Non-limiting exemplary automated scanning capillary viscometers useful in the invention include Hemathix Blood Analyzer (Health Onvector), Rheolog (Rheologics), and BVD (Bio-Visco).

Non-limiting exemplary rotating viscometers useful in the invention include Brookfield and Contraves.

In one embodiment, blood flow parameters can be measured and used in conjunction with measurements of complete blood count (CBC), fibrinogen, globulins, lipid profile, ankle/toe brachial index, pulse volume recording, and the like in order to assess the therapeutic outcome of the therapy.

A method according to the present invention allows for the improvement of the dosing of an antiplatelet agent for subjects in need thereof. In one embodiment, measurement or control of blood flow parameters is used as a marker for determining the appropriate dose of any compound administered to subjects having a disease or disorder associated with abnormal hemodynamic thrombogenicity in the treatment of the disease or disorder.

In order to monitor the treatment of a disease or disorder associated with abnormal hemodynamic thrombogenicity, measurements and/or control of whole blood viscosity is used to improve the safety and efficacy of the antiplatelet agent that is being administered to the subject, whereby the antiplatelet agent can be better managed and ideally optimized.

Another aspect of the present invention is the use of blood flow parameters of whole blood viscosity and percentage modulation in viscosity during treatment with an antiplatelet agent to monitor and control the safety, efficacy or dose of the antiplatelet agent.

Thus, in one embodiment according to the present invention, whole blood viscosity values may be used to assess the need for the administration and dosing of a desired antiplatelet agent. For example, the antiplatelet agent is thereby administered with the use of blood flow parameters as a means of monitoring and assessing the need for change in the dosage of the antiplatelet agent.

According to an embodiment of the present invention, the whole blood viscosity of a subject who is being treated with an antiplatelet agent is measured prior to receipt of a first dose of an antiplatelet agent in order to obtain an first blood viscosity value. After a period of time after the first dose, a second blood viscosity value is obtained. In some instances, the difference between the whole blood viscosity pre-first dose of the antiplatelet agent and post-first dose of the antiplatelet is determined, and if the difference in the whole blood viscosity is judged to have crossed a threshold value, the dosage of the antiplatelet agent can adjusted. Thus, the change in the whole blood viscosity due to a treatment modality that can cause changes in the whole blood viscosity such as treatment with an antiplatelet agent can be used to assess the need for modification of the dose of the antiplatelet agent.

As such, measurement of, control of and reductions in whole blood viscosity can be used in a method according to the present invention to treat a disease or disorder associated with abnormal hemodynamic thrombogenicity. This is done in conjunction with improved administration and dosing of an antiplatelet agent to a subject in need thereof.

In one embodiment, a low shear blood viscosity range of about 6 to 14 cP measured at a shear rate of 5 sec⁻¹ is associated with responsiveness to antiplatelet therapy. In another embodiment, a low shear blood viscosity range of about 7 to 12 cP measured at a shear rate of 5 sec⁻¹ is associated with responsiveness to antiplatelet therapy. In another embodiment, a low shear blood viscosity range of about 8 to 11 cP measured at a shear rate of 5 sec⁻¹ is associated with responsiveness to antiplatelet therapy.

In one embodiment, blood viscosity is used to optimize dosing of an antiplatelet agent, whereby efficacy of the antiplatelet agent may be improved.

In one embodiment, the invention includes monitoring a subject during an antiplatelet therapy regimen comprising determining the blood viscosity values of the subject and using the blood viscosity values to determine whether the levels of hemodynamic thrombogenicity are reduced by the platelet antagonist therapy, thereby indicating the production of a therapeutic effect.

In one embodiment of the invention, blood viscosity can be used to monitor and assess the efficacy and safety of an antiplatelet therapy regimen in a subject.

Thereby, blood viscosity is used as a marker to identify the optimal dose of an antiplatelet agent. In one embodiment, blood viscosity can be used to target the dose of the antiplatelet therapy regimen used to treat the disease or disorder associated with abnormal hemodynamic thrombogenicity in the subject.

Accordingly, the invention provides pre-treating the subject with an agent that modulates the blood viscosity to a range of 6 to 14 cP measured at a low shear rate of 5 sec⁻¹ and thereby converting the subject into one who will be optimally responsive to the antiplatelet therapy. In another embodiment, the invention provides pre-treating the subject with an agent that modulates the blood viscosity to a range of 7 to 12 cP measured at a low shear rate of 5 sec⁻¹ and thereby converting the subject into one who will be optimally responsive to the antiplatelet therapy. In another embodiment, the invention provides pre-treating the subject with an agent that modulates the blood viscosity to a range of 8 to 11 cP measured at a low shear rate of 5 sec⁻¹ and thereby converting the subject into one who will be optimally responsive to the antiplatelet therapy.

In one embodiment, a blood viscosity range of about 12 to 15 cP or greater measured at a low shear rate of 5 sec⁻¹, is associated with resistance to antiplatelet therapy. In one embodiment, the subject who is resistant to an antiplatelet therapy can become responsive to the antiplatelet therapy by treating the subject with an agent that produces a blood viscosity of about 12 cP or less when measured at a low shear rate of 5 sec⁻¹.

In one embodiment, blood viscosity value is used to optimize dosing of an antiplatelet agent, whereby efficacy of the antiplatelet agent may be improved. The optimized dosing of the antiplatelet agent to generate a blood viscosity value of about 12 cP or less when measured at a low shear rate of 5 sec⁻¹ contributes to improvements for health care and welfare of mankind such as prevention of hemostasis serving as an inducer for thrombosis, improvement of the active capacity in the peripheral tissue such as muscles concerning with increases in supplying oxygen and nutrients, improvement of hypertension along with the decrease of peripheral blood vessel resistance, prevention of arteriosclerosis progression and improvement of the conditions of heart failure caused by a decrease of peripheral blood vessel resistance.

Without wishing to be bound by any particular theory, it is believed that the blood viscosity value depicted in Table 1 provides blood viscosity ranges and thresholds applicable to the present invention.

TABLE 1
Blood Viscosity Ranges and Thresholds in Antiplatelet Therapy
[cP at SR 5 sec⁻¹]

| | | Target | |
|---|---|---|---|
| | Hemorrhagic Toxicity | Range | Resistance to Therapy |
| Broad | 5 | 6-14 | 15 |
| Medium | 6 | 7-12 | 13 |
| Narrow | 7 | 8-11 | 12 |

For example, in one instance, a blood viscosity value of about 5 cP or less when measured at a low shear rate of 5 sec⁻¹ corresponds to hemorrhagic toxicity whereas a blood viscosity value of about 6 to 14 cP when measured at a low shear rate of 5 sec⁻¹ corresponds to a therapeutic target range and a blood viscosity value of about 15 cP when measured at a low shear rate of 5 sec⁻¹ corresponds to resistance to therapy. In another instance, a blood viscosity value of about 6 cP or less when measured at a low shear rate of 5 sec⁻¹ corresponds to hemorrhagic toxicity whereas a blood viscosity value of about 7 to 12 cP when measured at a low shear rate of 5 sec⁻¹ corresponds to a therapeutic target range and a blood viscosity value of about 13 cP when measured at a low shear rate of 5 sec⁻¹ corresponds to resistance to therapy. In another instance, a blood viscosity value of about 7 cP or less when measured at a low shear rate of 5 sec⁻¹ corresponds to hemorrhagic toxicity whereas a blood viscosity value of about 8 to 11 cP when measured at a low shear rate of 5 sec⁻¹ corresponds to a therapeutic target range and a blood viscosity value of about 12 cP when measured at a low shear rate of 5 sec⁻¹ corresponds to resistance to therapy.

### Indication for Adjusting or Maintaining Drug Dose

An indication for adjusting or maintaining a subsequent drug dose can be based on the increase or decrease of the whole blood viscosity value. For example, an indication for adjusting or maintaining a subsequent drug dose often is based on the value of whole blood viscosity. In some instances, indication for adjusting or maintaining a subsequent drug dose is based on whole blood viscosity in combination with any other criteria including but is not limited to complete blood count (CBC), fibrinogen, globulins, lipid profile, ankle/toe brachial index, pulse volume recording, and the like. The measurement of blood viscosity and other assessment criteria is not limited to one time, but may be performed at a plurality of times after the initial dose of the antiplatelet agent.

An indication for adjusting or maintaining a subsequent drug dose often is generated by comparing a determined level of whole blood viscosity in a subject to a predetermined level of whole blood viscosity. A predetermined level of whole blood viscosity sometimes is linked to a therapeutic or efficacious amount of drug in a subject, sometimes is linked to a toxic level of a drug, sometimes is linked to presence of a condition, sometimes is linked to a treatment midpoint and sometimes is linked to a treatment endpoint, in certain embodiments. A predetermined level of a whole blood viscosity level sometimes includes time as an element, and in some embodiments, a threshold is a time-dependent signature.

Some treatment methods comprise (i) administering a drug to a subject in one or more administrations (e.g., 1, 2, 3, 4, 5, 6, 7, 8, 9 or 10 doses), (ii) determining the whole blood viscosity value in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, whole blood viscosity value is determined after each dose of drug has been administered to the subject, and sometimes whole blood viscosity is not determined after each dose of the drug has been administered (e.g., blood viscosity is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication. Thus, a clinician can opt to adjust or not adjust a subsequent drug dose based on the indication provided.

An indication for adjusting a drug dose or subsequent drug dose can be carried out by adjusting the dose of the drug or the schedule of the dosing. Thus, an indication for adjusting or maintaining a subsequent drug dose schedule can be considered by a clinician, and the clinician may act on the indication in certain embodiments. In some embodiments, a clinician may opt not to act on an indication. Thus, a clinician can opt to adjust or not adjust a drug dose schedule or subsequent drug dose schedule based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a whole blood viscosity threshold may be provided in a table, and a clinician may compare the whole blood viscosity value determined for a subject to the threshold. The clinician then can identify from the table an indication for subsequent drug dose. In certain embodiments, an indication can be presented (e.g., displayed) by a computer after whole blood viscosity value is provided to computer (e.g., entered into memory on the computer). For example, whole blood viscosity value determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

### Administration/Dosage/Formulations

The regimen of administration may affect what constitutes an effective amount. The therapeutic formulations may be administered to the subject either prior to or after the onset of abnormal platelet function, hemodynamic thrombogenicity, and related conditions. Further, several divided dosages, as well as staggered dosages may be administered daily or sequentially, or the dose may be continuously infused, or may be a bolus injection. Further, the dosages of the therapeutic formulations may be proportionally increased or decreased as indicated by the whole blood viscosity value and may include indications of exigencies of the therapeutic or prophylactic situation as measured.

Administration of the compositions of the present invention to a subject, preferably a mammal, more preferably a human, may be carried out using known procedures, at dosages and for periods of time effective to treat diseases and disorders associated with abnormal platelet function and hemodynamic thrombogenicity in the subject. An effective amount of the therapeutic compound necessary to achieve a therapeutic effect may vary according to the whole blood viscosity and an some instances factors such as the state of the disease or disorder in the subject; the age, sex, and weight of the subject. Dosage regimens may be adjusted using at least whole blood viscosity value as a marker to provide the optimum therapeutic response. Based on the disclosure presented herein, one of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired whole blood viscosity value is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of abnormal platelet aggregation and hemodynamic thrombogenicity and related conditions in a subject.

In one embodiment, the compositions encompassed in the invention are formulated using one or more pharmaceutically acceptable excipients or carriers. In one embodiment, the pharmaceutical compositions encompassed in the invention comprise a therapeutically effective amount of a desired compound and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin. In one embodiment, the pharmaceutically acceptable carrier is not DMSO alone.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any subject is determined by the attending physical taking all other factors about the subject into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a desired compound is from about 1 mg and about 2,500 mg. In some embodiments, a dose of a desired compound is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of KSHV infection and related conditions. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends at least on whole blood viscosity of the subject. In some instances the effective amount or dose of the drug depends on age, sex and weight of the subject, the current medical condition of the subject and the progression of the abnormal hemodynamic thrombogenicity and related conditions in the subject being treated. The skilled artisan armed with the present disclosure is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the subject's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the subject's conditions has occurred, a maintenance dose is administered if necessary. Subsequently, the dosage or the frequency of administration, or both, is reduced, as a function of the viral load, to a level at which the improved disease is retained. In one embodiment, subjects require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Active compounds exhibiting high therapeutic indices are preferred. The data obtained from cell culture assays and animal studies are optionally used in formulating a range of dosage for use in human. The dosage of such active compounds lies preferably within a range of circulating concentrations that include the ED₅₀ with minimal toxicity. The dosage optionally varies within this range depending upon the dosage form employed and the route of administration utilized.

Those skilled in the art recognizes or is able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental example. This example is provided for purposes of illustration only, and is not intended to be limiting unless otherwise specified. Thus, the invention should in no way be construed as being limited to the following example, but rather, should be construed to encompass any and all variations which become evident as a result of the teaching provided herein.

Without further description, it is believed that one of ordinary skill in the art can, using the preceding description and the following illustrative example, make and utilize the compounds of the present invention and practice the claimed methods. The following working example therefore, specifically point out the preferred embodiments of the present invention, and are not to be construed as limiting in any way the remainder of the disclosure.

### Example

### Comparison of Ticagrelor Against Clopidogrel and Aspirin on Blood Viscosity in Peripheral Artery Disease Patients

Ticagrelor is a cyclopentyltriazolopyrimidine that has been shown to reduce significantly the rate of cardiovascular disease (CVD) events and death compared with clopidogrel in 18,624 patients having prior acute coronary syndrome (Wallentin et al. 2009 N Engl J Med 361:1045-57). The adenosine diphosphate receptor P2Y12 antagonist, clopidogrel 75 mg was more effective than aspirin 325 mg in reducing CVD events or death in a clinical trial of 19,185 patients with atherosclerosis, presenting with symptomatic peripheral artery disease (PAD), recent ischemic stroke or myocardial infarction (MI) (CAPRIE Steering Committee, 1996 Lancet 348:1329-39). In a pre-specified analysis of risk by the type of vascular events at entry, there was no reduction in MI, stroke or cardiovascular death rates in patients who entered the trial with an ischemic stroke or MI while there was a relative risk reduction of 23.8% (p=0.0028) in the group entering the trial with symptomatic PAD. The disproportionate event reduction in patients with PAD patients suggests that mechanisms other than atherosclerosis or thrombosis may contribute to CVD event reduction in these patients. Without wishing to be bound by any particular theory, it is believed that blood rheology is an important factor that influences macrovascular and microvascular flow in lower extremity arteries, and predicts CVD events in PAD patients.

A previous study comparing 120 patients having intermittent claudication with normal age-matched controls found blood viscosity was significantly higher among claudicants (p<0.001) with the greatest difference in blood viscosity observed at lowest shears (Dormandy et al., 1974 Proc R Soc Med 67:446-7). At high shear rates, patients with blood viscosity above 4.5 cP had mean claudication distance of 126 meters compared to 289 meters for patients with high-shear viscosity below that threshold. This report suggested the use of the term rheological claudication to describe approximately 25% of moderate to severe claudicants with hyperviscosity of blood having significantly worse prognoses.

In a random sample of 1,581 men and women 55 to 74 years of age with symptomatic or asymptomatic PAD, whole blood viscosity and fibrinogen were independently associated with peripheral arterial narrowing (Lowe et al., 1993 Circulation 87:1915-20). Plasma viscosity was also associated with claudication. The risk of claudication for patients in the upper quintile of plasma viscosity was 3.4 times greater than the risk for those in the lowest plasma viscosity quintile. The authors implicated blood rheologic factors in the pathogenesis of lower limb ischemia in the general population.

The materials and methods employed in this experiment are now described.

An experiment was designed to include a double-blind, placebo-controlled design study to examine and compare the effects of low-dose aspirin, clopidogrel, and ticagrelor on blood viscosity in patients with PAD. Study participants are randomized into 3 groups and are eligible to participate if they have ankle-brachial index less than or equal to 0.85. Aspirin 81 mg has been shown to have no significant effect on blood viscosity in healthy individuals (Rosenson et al., 2008 Microcirculation 15:615-20), and is used as control.

The Experiment is designed to compare the effects of aspirin, clopidogrel, and ticagrelor in a double-blind, randomized study design on blood viscosity at both low (5 s⁻¹) and high (300 s⁻¹) shear rates.

If a reduction in blood viscosity is determined, the implications of this research study would be to provide a rationale for the following: (1) improvement of microvascular flow abnormalities in PAD patients by ticagrelor, an antiplatelet therapy; (2) use of blood viscosity changes to monitor and guide antiplatelet therapy; (3) prognostic role for blood viscosity changes on cardiovascular and microcirculatory events in PAD patients.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for determining the efficacy of an antiplatelet agent in a subject, the method comprising:
monitoring at least one blood flow parameter in a subject;
administering an antiplatelet agent at a first dose to a subject;
and targeting a new dose of the agent if the value of the at least one blood flow parameter crosses a predetermined threshold value.

2. The method of claim 1 wherein the at least one blood flow parameter is selected from the group consisting of whole blood viscosity, low shear viscosity, and yield stress of blood.

3. The method of claim 1, wherein the subject is suffering from a vascular disease or disorder.

4. The method of claim 3, wherein the vascular disease or disorder is selected from the group consisting of a disorder of hemodynamic thrombogenicity, a thrombotic disorder, ischemia, acute coronary syndrome, stroke, ischemic complications of peripheral vascular disease, deep vein thrombosis, myocardial infarction, coronary artery disease, cerebrovascular disease, peripheral arterial disease, diabetes mellitus, diabetic retinopathy, atrial fibrillation, congestive heart failure, pulmonary embolism, and any combination thereof.

5. The method of claim 1, wherein the antiplatelet agent is ticagrelor.

6. The method of claim 1, the at least one blood flow parameter is whole blood viscosity further wherein the whole blood viscosity value of less than about 13 cP when measured at a low shear rate of 5 sec⁻¹ indicates the efficacy of the antiplatelet agent for the subject.

7. A method for improving the safety and efficacy of an antiplatelet agent in a subject, the method comprising:
measuring a whole blood viscosity of the subject to obtain a first value;
administering an antiplatelet agent to the subject;
measuring a whole blood viscosity of the subject to obtain a second value wherein a value of less than about 13 cP but greater than about 6 cP when measured at a low shear rate of 5 sec⁻¹, indicates the hemorrhagic safety and thrombogenic efficacy of the antiplatelet agent for the subject;
and administering a subsequent dose of the antiplatelet agent to the subject to attain the value of less than about 12 cP when measured at a low shear rate of 5 sec⁻¹.

8. The method of claim 7, wherein the subject is suffering from a vascular disease or disorder.

9. The method of claim 8, wherein the vascular disease or disorder is selected from the group consisting of a disorder of hemodynamic thrombogenicity, a thrombotic disorder, ischemia, acute coronary syndrome, stroke, ischemic complications of peripheral vascular disease, deep vein thrombosis, myocardial infarction, coronary artery disease, cerebrovascular disease, peripheral arterial disease, diabetes mellitus, diabetic retinopathy, atrial fibrillation, congestive heart failure, pulmonary embolism, and any combination thereof.

10. The method of claim 7, wherein the antiplatelet agent is ticagrelor.

11. A method for improving the safety and efficacy of an antiplatelet agent in a subject, the method comprising:
measuring a whole blood viscosity of the subject to obtain a first viscosity value;
administering an antiplatelet agent to the subject;
measuring a whole blood viscosity of the subject to obtain a second viscosity value;
determining a value for a difference between the first viscosity value and the second viscosity value;
comparing the value for the difference to a threshold value;
targeting a whole blood viscosity value for the subject if the value of the difference has crossed the threshold value;
and administering a subsequent dose of the antiplatelet agent to attain the targeted whole blood viscosity.

12. The method of claim 11, wherein the subject is suffering from a vascular disease or disorder.

13. The method of claim 12, wherein the vascular disease or disorder is selected from the group consisting of a disorder of hemodynamic thrombogenicity, a thrombotic disorder, ischemia, acute coronary syndrome, stroke, ischemic complications of peripheral vascular disease, deep vein thrombosis, myocardial infarction, coronary artery disease, cerebrovascular disease, peripheral arterial disease, diabetes mellitus, diabetic retinopathy, atrial fibrillation, congestive heart failure, pulmonary embolism, and any combination thereof.

14. The method of claim 11, wherein the antiplatelet agent is ticagrelor.
